(19) United States
(12) Patent Application Publication
(10) Pub. No.: US 2018/0264220 A1
(43) Pub. Date: Sep. 20, 2018

(54) Seal Between Bag Valve Mask and Face, with Nasal Cannula

(71) Applicant: STC.UNM, Albuquerque, NM (US)

(72) Inventor: Hans Fredrick Hurt, Albuquerque, NM (US)

(73) Assignee: STC.UNM, Albuquerque, NM (US)

(21) Appl. No.: 15/753,887

(22) PCT Filed: Aug. 22, 2016
(86) PCT No.: PCT/US2016/047983

§ 371 Date: Feb. 20, 2018

Related U.S. Application Data
(60) Provisional application No. 62/208,109, filed on Aug. 21, 2015.

(51) Int. Cl. A61M 16/06; A61M 39/08

(52) U.S. Cl. CPC A61M 16/0605; A61M 39/08; A61J 15/0003; A61M 2202/0208; A61M 2210/0618; A61M 16/0672

(57) ABSTRACT

A medical device sealing system for use with a positive pressure ventilation mask and a nasal cannula. The system includes a plurality of sealing elements. The sealing elements include an opening sized to receive a nasal cannula. The sealing elements are spaced apart a distance sufficient to cause the sealing elements to engage the edges of a mask and to form a seal between the edges of a mask and a patient.

### BACKGROUND OF THE INVENTION

A nasal cannula is a way to administer oxygen to patients via small tubing that blows oxygen into the nose via two prongs as shown in FIG. 1.

There is growing use of a nasal cannula combined with a bag valve mask during the pre-oxygenation time period before intubation. Weingard and Levitan published an article in 2012 that summarized the benefits of having a nasal cannula in place during the apneic period during airway manipulation and endotracheal intubation.

Placement of a nasal cannula often happens before sedatives and paralytics are given to a patient. After the patient is given medications, the patient becomes apneic, stops breathing and ceases to move air in and out of the lungs. The lungs continue to absorb oxygen at about approximately 200 ml/min and expel CO₂ at 60 ml/min in an adult. This creates a small negative pressure, approximately 140 ml/min, that draws gas into the lungs from the mouth and nose, the nasal cannula fills the back of the mouth with oxygen so that the gas pulled into the lungs is closer to 100% oxygen then normal room air gas that is 21% oxygen. This helps to prolong the time to hypoxia in a patient who is not breathing.

In some situations, patients have very poor lung function and become hypoxic (low blood oxygen levels) very quickly after sedatives and paralytics are given. At that time, positive pressure ventilation with a bag valve mask is started, which pushes fresh oxygenated gas into the lungs and improves the blood oxygen levels.

Positive pressure ventilation with a bag valve mask (BVM) can be done with a nasal cannula in place, but it can be challenging to achieve a good mask to face seal around the tubing of the nasal cannula. If there is a compromised mask to face seal, it can be difficult to push air into the lungs, and might not be possible to hold positive end expiratory pressure (PEEP, pressure held in the lungs to keep the lungs tented open with gas at the end of the exhalation phase of breathing) or hold end inspiratory pressure to tent open a patient's airway with the lungs fully expanded. A leak can also allow anesthetic gases, which may be flammable, to escape into the operating room. This has the potential to create safety and health issues in the operating room and for the operating room staff.

Due to the difficulty in certain situations of creating a sufficient mask to face seal during positive pressure ventilation, some medical practitioners are hesitant to use a nasal cannula during the intubation time period even though there are proven benefits for its placement during the apneic time of airway manipulation after the BVM is removed. Placing the nasal cannula after the BVM is removed takes up limited time during the intubation and apneic period that some patients do not have to spare.

Other situations outside of the intubation period when a mask to face seal is important with positive pressure ventilation is with Non Invasive Positive Pressure Ventilation (NIPPV), such as CPAP and BIPAP (Continuous Positive Airway Pressure and Biphasic Positive Airway Pressure). People use NIPPV who have obstructive sleep apnea (OSA) and other lung problems, such as infection or chronic pulmonary diseases that compromise the function of their lungs to the point of needing pressure support but not clearly indicating intubation. In the hospital these people may have the need for a nasogastric tube (a tube entering the nose and going to the stomach) to aid in suctioning or supplying medications or nutrients to the stomach. The nasogastric tube is also a circular tubing that makes it difficult to have a good mask to face seal.

### BRIEF SUMMARY OF THE INVENTION

In one embodiment, the present invention provides an improved nasal cannula tubing design that improves the mask to face seal around the nasal cannula tubing.

In other embodiments, the present invention provides a patch design which may be shaped, pliable and/or rubber gummy patches that wrap around the tubing used with existing nasal cannulas to improve the mask to face seal.

In yet other embodiments, the present invention improves the mask to face seal while using tubing by providing a seal that is retained in place by using a soft and pliable gel or hydrocolloid material.

In yet other embodiments, the present invention provides a device that improves the safety and practicality of its use. Specifically, the designs of the present invention improve the specific interaction of the bag valve mask over the nasal cannula allowing for a more easily achievable airtight seal aiding in the effectiveness of positive pressure ventilation.

In still further embodiments, the pliable patches of the present invention aid in improving the mask to face seal in situations needing NIPPV with a nasal cannula or when a nasogastric tube is in place. Often times when a nasogastric tube is used, the mask to face seal is poor causing a significant air leak that requires higher pressure of the mask onto the face. This higher pressure can cause skin ulcerations and erosions from the constant force of the mask.

Additional objects and advantages of the invention will be set forth in part in the description which follows, and in part will be obvious from the description, or maybe learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 illustrates a conventional nasal cannula that is used to administer oxygen to patients via small tubing that blows oxygen into the nose via two prongs.

FIG. 2 illustrates an embodiment of the present invention that uses patches or seals that are designed to eliminate the leakage around the edges of a standard nasal cannula tubing design when used in connection with a mask.

FIG. 3 is an angled top view of the embodiment of the present invention shown in FIG. 2.

FIG. 4 is a cross-section view of the embodiment of the present invention shown in FIG. 2.

FIG. 5 illustrates how a seal is formed by the embodiment of the present invention shown in FIG. 2.

FIG. 6 illustrates another embodiment of the present invention that uses interconnected patches or seals that are designed to eliminate the leakage around the edges of a standard nasal cannula tubing design when used in connection with a mask.

FIG. 7A and FIG. 7B demonstrate an embodiment of the current invention that has a containment device and an adhesive overlay, that secures the tubing in place and secures the overlapping seal.

FIG. 8A and FIG. 8B illustrate how a seal is formed by an embodiment of the present invention.

FIG. 9 illustrates another embodiment of the present invention that forms a bridge network that is designed to eliminate the leakage around the edges of a standard nasal cannula tubing design when used in connection with a mask.

FIG. 10 is a side view of the embodiment of the present invention shown in FIG. 9.

FIG. 11 is a front view of the embodiment of the present invention shown in FIG. 9.

FIG. 12 is a side view of the embodiment of the present invention shown in FIG. 9 used with a mask.

FIG. 13 illustrates another embodiment of the present invention that provides tubing having contoured sections to improve the seal between a positive pressure mask and a patient.

FIG. 14 provides a front view of another embodiment of the present invention configured for use with a nasogastric tube.

FIG. 15 is a side view of the embodiment shown in FIG. 14.

FIG. 16 is a side view of the embodiment shown in FIG. 14 used with a nasogastric tube and mask.

FIG. 17 is a front view of the embodiment shown in FIG. 14 used with a nasogastric tube and mask.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

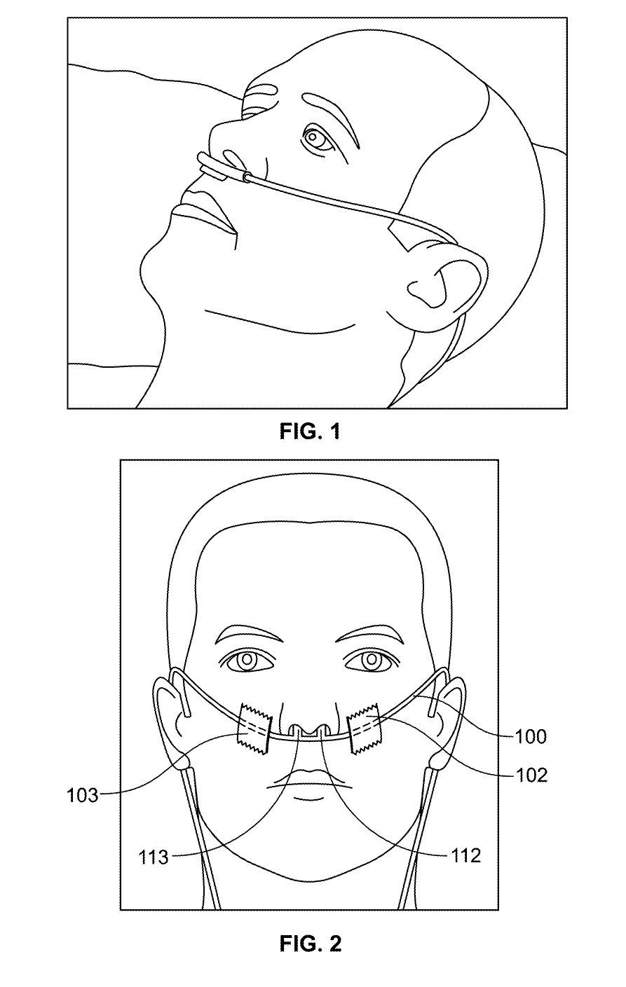

The present invention replaces the prior art circular nasal cannula tubing with designs that use tubing with cross-sectional shapes thereon that improve the seal between the mask and the patient. In one embodiment, as shown in FIG. 2, the tubing of the main delivery hose 100 has attached thereto sealing elements 102 and 103 that are spaced apart and located outwardly from nasal tubes 112-113.

Sealing elements 102 and 103 are located approximately where the mask would be fitted to the patient and are configured to present a shape that acts to create an airtight or substantially airtight seal between the mask and patient. In yet other embodiments the sealing elements are configured to be movable along the tubing so as to accommodate masks of varying sizes.

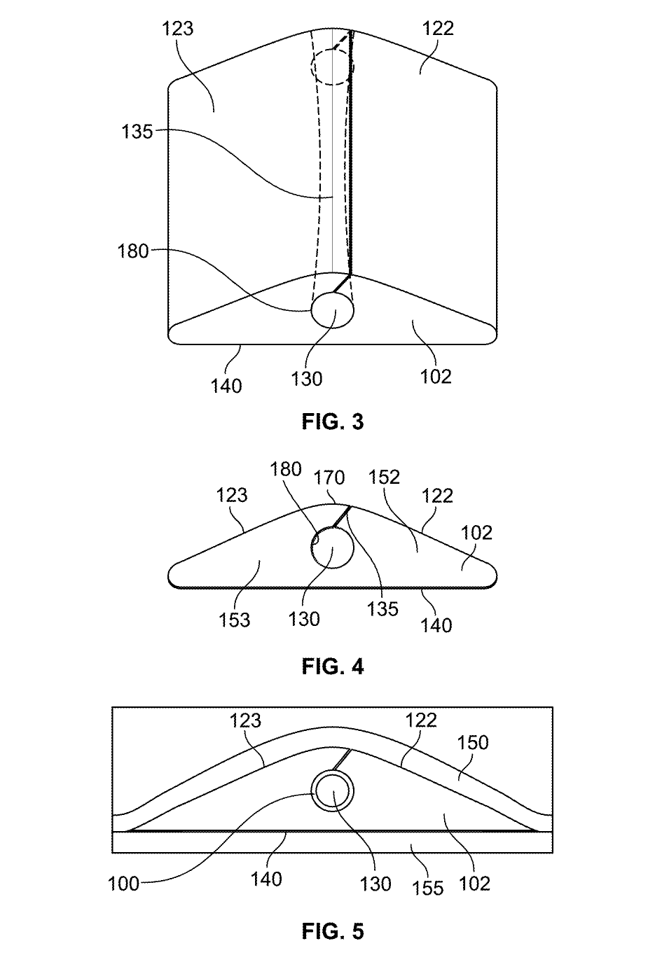

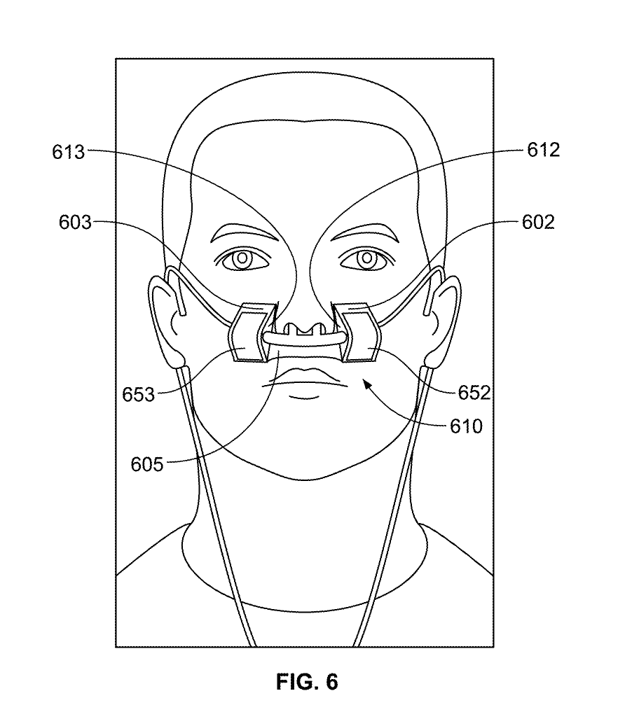

As shown in FIGS. 3-5, in a preferred embodiment, sealing elements 102 and 103 may be configured to form a flattened shape that may be ovaloid in design. As is also shown, in yet another embodiment, sides 122 and 123 maybe tapered to improve the seal as well. As is also shown, a passageway or central opening 130 is also provided to contain tubing 100 for the delivery of gas. Opening 130 may be configured to mimic the shape of the outer surface of the tube or it may have an independent shape that is flattened as well.

In still further embodiments, an access slit 135 may be provided or formed in sealing elements 102 and 103, such as by cutting the sleeve at angle to form overlapping sections 152 and 153 that aid in the compatibility of various size tubing while maintaining a smooth superficial surface for the mask to be sealed.

In yet other embodiments, as shown in FIGS. 3-5, surface 140 of sealing elements 102 and 103 engages patient 155. Surface 140 may be anatomically shaped, such as in the form of a flat surface, contoured, molded or pliable surface, to match that of the patient more closely. Surface 140 may also include adhesive for affixing the sealing elements to a patient. Surfaces 122 and 123 may be configured to be flattened or complimentary to the edge of mask 150.

In another preferred embodiment, the cross sectional plane of the seals may be triangular. In other embodiments, surfaces 122 and 123 may be in the form of a curved or flattened peak 170, as shown in FIG. 4, for a smooth top to allow for an easy mask to patch seal.

Other embodiments include a tubular sleeve 180, as shown in FIGS. 3 and 4, through the middle of the patch where the nasal cannula tubing or nasogastric tubing would be enveloped. Sleeve 180 may have a slight hourglass shape, which allows for easy moldability for multiple size tubing diameters. Sleeve 180 may also have a tapered design, larger diameter medially to smaller laterally as nasal cannulas have various tubing sizes around the prongs that dispense the oxygen which is often a larger size. The patches may be placed on the face immediately lateral to the nose on the right and left under the nose in a location where the nasal cannula normally lies.

Sealing elements 102 and 103 aid in holding the nasal cannula in place as well as improving the mask to face seal. The material may be pliable, or be soft and flexible so it conforms around the tubing and to the contours of the face and mask easily, and be of a substance that does not allow air to move through it. The material may also have a memory to an initial molded or cut shape and form. Materials that may be used include a solid gel padding or hydrocolloid materials.

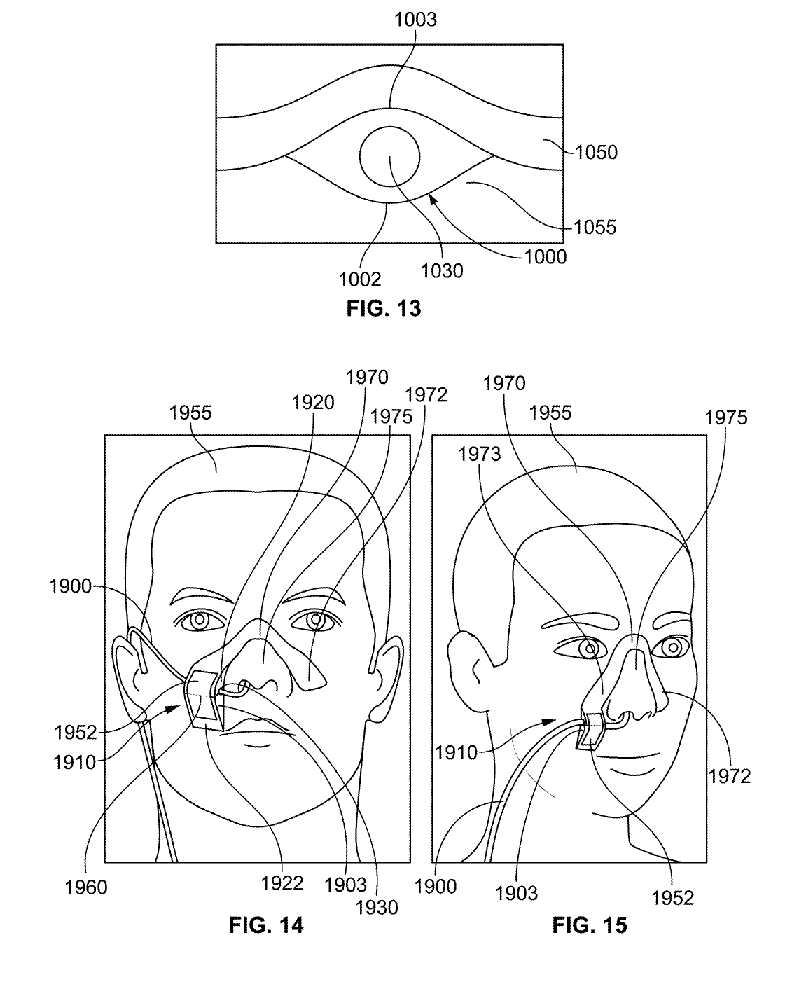

In other embodiments of the present invention, as shown in FIG. 13, instead of providing tabs, inserts, sealing elements or patches that are affixed to the tubing, the tubing itself may be designed to eliminate the leakage around the edges of a standard nasal cannula tubing design when used in connection with a mask. For these embodiments of the present invention, the tubing may have a bottom, such as a flat surface or be of the designs described above and shown in FIGS. 2-5 to form the sealing elements that are spaced apart and separated from the nasal prongs.

In yet other embodiments, the present invention provides designs that aid in placing the seals and mask onto a patient. As shown in FIG. 6, this embodiment provides spaced apart sealing elements or components 602 and 603 connected by segment 605 to form pad 610 which is configured to be placed under the nose of a patient. In a preferred embodiment, segment 605 may be a thin piece of material that would also have an elastic ability to stretch to change length so as to fit different widths of the nose. This connection between the two sealing elements or lateral components 602 and 603 makes it easier to place pad 610 as a single unit and it increases the overall size so there is less chance of it falling into the mouth or nose of a patient. In yet other embodiments, sealing elements or components 602 and 603 include sidewalls 612 and 613 which may be urged inwardly or towards each other against the nostrils by segment 605 to engage the nostrils to form a friction fit that assists in holding pad 610 in place.

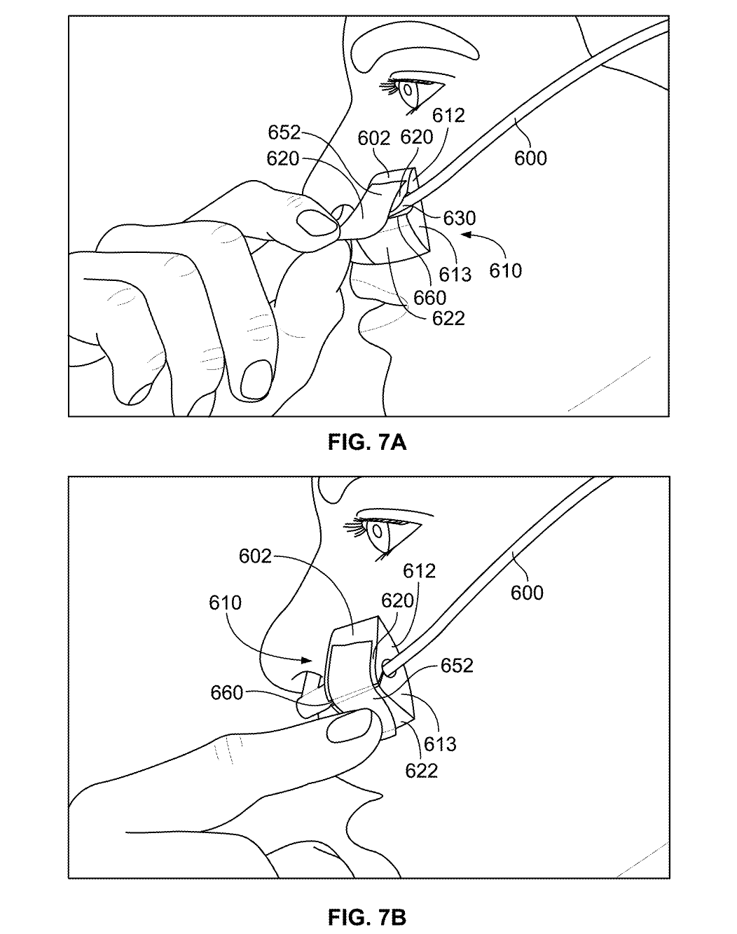

As shown in FIGS. 6, 7A and 7B, in yet other embodiments, the present invention provides securement straps 652 and 653. The straps aid in retaining nasal cannula 600 within pad 610 in opening 630. In a preferred embodiment, each pad or seal includes a slit that creates two sections in each pad that are urged together to form an improved seal.

For sealing element 602 this arrangement is shown in FIGS. 7A and 7B. Sealing element 603 may be configured in a similar manner. As shown, slit or opening 660 forms a first section 620 that is hinged towards section 622 by strap 652. This causes sealing element 602 to deform around tube 600 thereby producing an improved seal around the tubing. In yet other embodiments, pad 610 is configured to be made of a deformable material that is urged against the tubing and patient to form a seal.

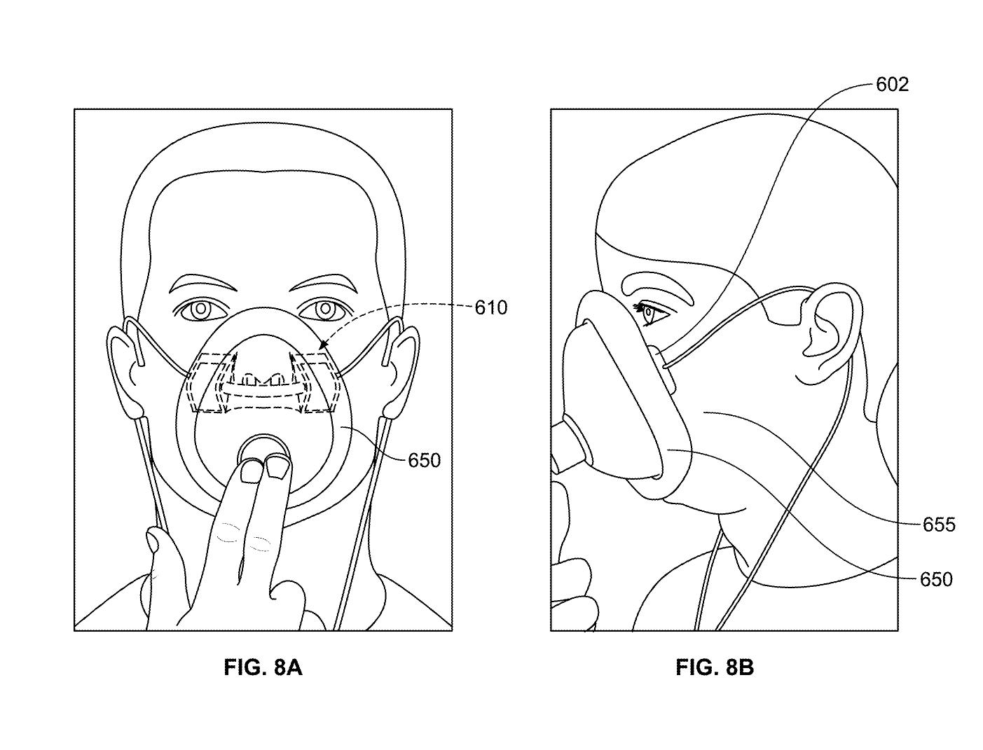

In use, as shown in FIGS. 8A-8B, pad 610 is placed on a patient and then mask 650 is placed into position where the edges of the mask engage the sealing elements. As shown in FIG. 8B, sealing element 602 of pad 610 deforms against mask 650 and patient 655 to form a seal caused by the compression of pad 610 that prevents gas from escaping during use.

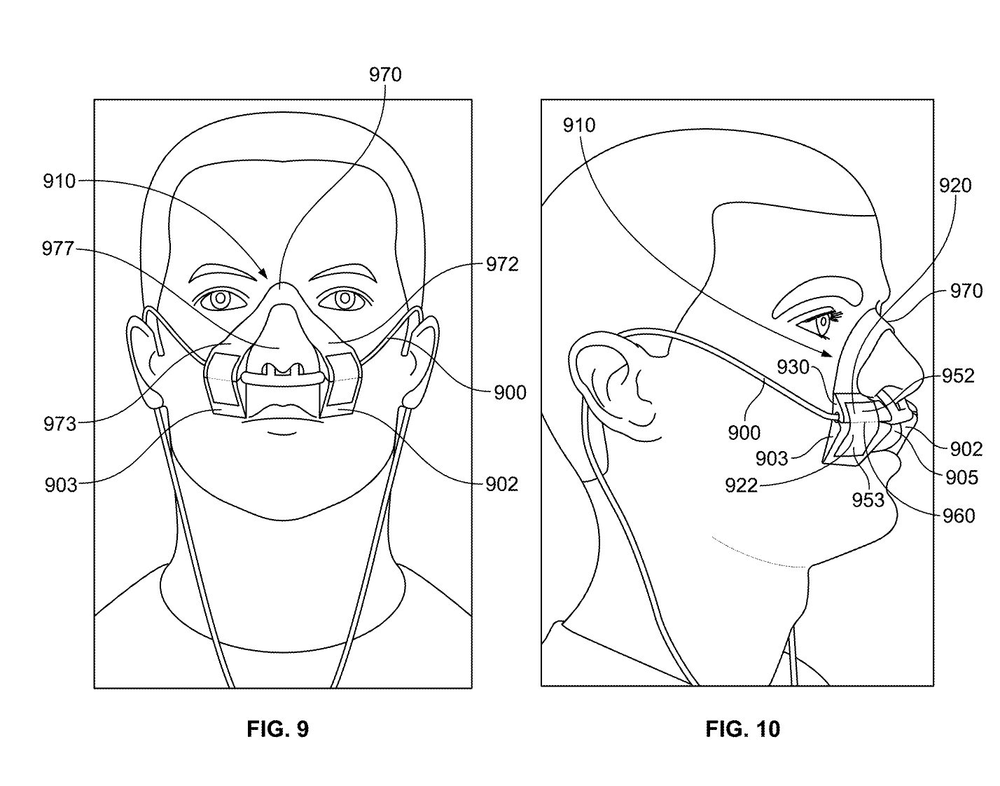

In yet other embodiments of the present invention, as shown in FIG. 9, pad 910 may also include a bridge 970 that connects sealing elements or components 902 and 903 by arms 972 and 973 to form a triangular shaped pad adapted to go over the bridge of a nose 977 and fit around the nose. Bridge 970 has the benefit of making it easier to place pad 910 as a single unit and also makes pad 910 larger which reduces the aspiration risk of the smaller individual units. In addition, bridge 970 may also be used with the embodiments described above. In a preferred embodiment, bridge 970 may be combined with the embodiment of the present invention illustrated in FIG. 6.

Providing padding over the bridge of the nose may also be used in applications where the tube sealing slot was used with a nasogastric tube and with a bi-level positive airway pressure (BIPAP) mask or a continuous positive airway pressure (CPAP) mask. BIPAP and CPAP masks can create pressure over time on the bridge of the nose that can irritate and erode the skin. Pad 910 reduces the nasal bridge skin irritation associated with either CPAP or BIPAP masks.

As shown in FIG. 10, in yet another embodiment, pad 910 may include spaced apart sealing elements or components 902 and 903 connected by a linear segment 905 which is configured to be placed under the nose of a patient. In a preferred embodiment, segment 905 may be a thin piece of material that would also have an elastic ability to stretch to change length so as to fit different widths of the nose. This connection between the sealing elements or lateral components 902 and 903 makes it easier to place pad 910 as a single unit and it increases the overall size so there is less of a chance of it falling into the mouth or nose.

Securement straps, such as 953 for sealing element 903, may also be use with this embodiment. Sealing element 902 may be configured in a similar manner.

As shown in FIG. 10, the strap aids in retaining nasal cannula 900 within pad 910 in opening 930. In a preferred embodiment, each sealing elements includes a slit that creates two sections in each element that are urged together to form an improved seal.

For sealing element 903, slit or opening 960 forms a first section 920 that is hinged towards section 922 by strap 953. This causes sealing element 903 to deform around tube 900 thereby producing an improved seal around the tubing. In yet other embodiments, pad 910 is configured to be made of a deformable material that is urged against the tubing and patient to form a seal.

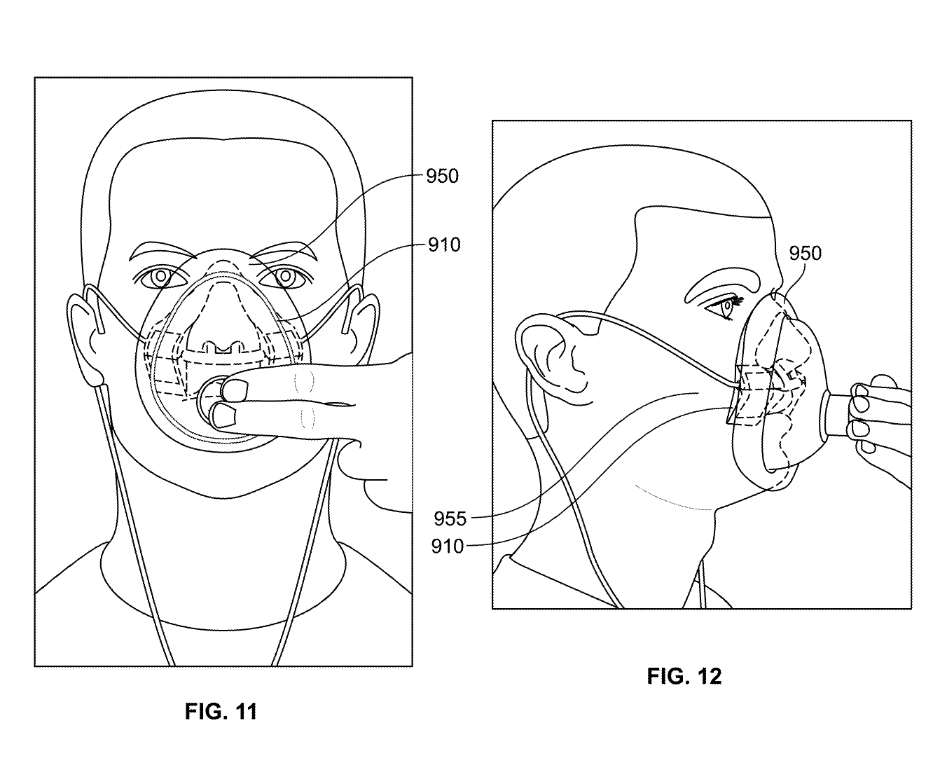

In use, as shown in FIGS. 11-12, pad 910 is placed on a patient and then mask 950 is placed into a location so that the other edges engage the sealing elements. As shown in FIG. 12, compressive forces cause pad 910 to deform against mask 950 and patient 955 to form a seal that prevents gas from escaping during use.

In still further embodiments of the present invention, tubing 1000 may also be configured to have one or more sections that are ovaloid, a tapered flattened shape as well as complimentary in shape to the edges of a mask, where the seals are located. As shown in FIG. 13, in cross-section, a preferred embodiment provides contoured sections having tapered edges 1002 and 1003 and an opening 1030. This configuration, as well as the configurations for the other embodiments described above and which may be used to configure tubing 1000, allow for a more complete seal between mask 1050 and patient 1055, if not a complete seal, when applying the positive pressure ventilation mask. The current circular tubing used in practice makes it difficult to accomplish a complete air tight seal as the mask and skin do not come in complete contact around the edges of the nasal cannula tubing leaving space for gas to escape when positive pressure is applied. The embodiments of the present invention may be used from the main supply hose to the nasal prongs, and be placed in alignment with the prongs so the tubing aligns to be flatter against the skin when the prongs are in a proper position in the nose.

The overall dimensions of the patches or contoured sections of tubing described above may also be configured to accommodate masks of various sizes. In addition, as described above, the seals may also be configured to be moveable along the tubing to accommodate different sized masks.

In addition, the embodiments described above may be used with a nasogastric tube. The patch sizes may be constructed in sizes that best fit the mask sizes for neonates, children, small adults, average adults and large adults. As mask diameter and shape will vary due to facial sizes, the patches may need to have multiple sizes to accommodate the appropriate size face and mask and to optimize the mask-face-tube seal.

In yet other embodiments of the present invention, as shown in FIGS. 14-17, pad 1910 is provided which includes a bridge 1970 that connects sealing element or component 1903 by arms 1972 and 1973 to form a triangular shaped pad adapted to go over the bridge of a nose 1975 and fit around the nose. Bridge 1970 has the benefit of making it easier to place pad 1910 as a single unit and also makes pad 1910 larger which reduces the aspiration risk of the smaller individual units. In addition, bridge 1970 may also be used with the embodiments described above.

Providing padding over the bridge of the nose may also be used in applications where the tube sealing slot was used with a nasogastric tube 1900 and with a bi-level positive airway pressure (BIPAP) mask or a continuous positive airway pressure (CPAP) mask. BIPAP and CPAP masks can create pressure over time on the bridge of the nose that can irritate and erode the skin. Pad 1910 reduces the nasal bridge skin irritation associated with either CPAP or BIPAP masks.

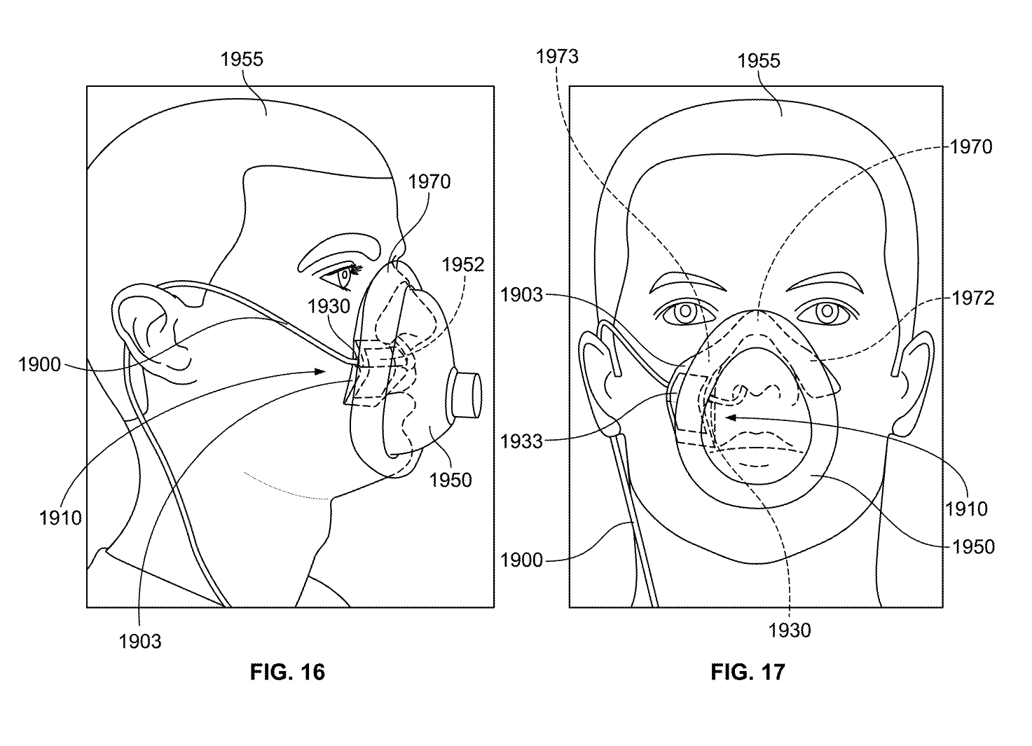

As shown in FIGS. 16 and 17, mask 1950 is placed over pad 1910 while nasogastric tube 1900 is located in opening 1930. As shown, sealing element 1903 of pad 1910 deforms against mask 1950 and patient 1955 to form a seal caused by the compression of pad 1910 that prevents gas from escaping during use.

As shown in FIGS. 14-17, in yet other embodiments, the present invention provides securement strap 1952. The strap aids in retaining nasogastric tube 1900 within pad 1910 in opening 1930. In a preferred embodiment, each pad or seal includes a slit that creates two sections in each pad that are urged together to form an improved seal.

For sealing element 1903 this arrangement is shown in FIG. 14. As shown, slit or opening 1960 forms a first section 1920 that is hinged towards section 1922 by strap 1952. This causes sealing element 1903 to deform around tube 1900 thereby producing an improved seal around the tubing. In yet other embodiments, pad 1910 is configured to be made of a deformable material that is urged against the tubing and patient to form a seal.

In yet other embodiments configured for use with a nasogastric tube, the diameter of the patch tube channel 1920 may also need to have variable diameters as there are multiple size nasogastric tubes and nasal cannulas to fit patients ranging from neonates to large adults, as well as multiple variations of feeding tubes with varying diameters.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. A medical device for delivering a gas to a patient for use with a positive pressure ventilation mask comprising: elongated tubing having a plurality of sealing elements, said sealing elements spaced apart a distance sufficient to cause said sealing elements to engage the edges of a mask and to form a seal between the edges of a mask and a patient.

2. The device of claim 1 wherein said sealing elements are comprised of a compressible material that forms a seal between a mask and a patient.

3. A medical device sealing system for use with a positive pressure ventilation mask and a nasal cannula comprising: a plurality of sealing elements, said sealing elements including an opening sized to receive a nasal cannula; and said sealing elements spaced apart a distance sufficient to cause said sealing elements to engage the edges of a mask and to form a seal between the edges of a mask and a patient.

4. The device of claim 3 wherein said sealing elements have a cross-sectional shape having a flattened edge connected to opposing tapered edges.

5. The device of claim 3 wherein said sealing elements have a cross-sectional shape having a flattened edge connected to opposing tapered edges, said tapered edges are complimentary in shape to the edge of a mask.

6. The device of claim 3 wherein said sealing elements have a triangular cross sectional shape with a curved top.

7. The device of claim 3 wherein a tubular sleeve forms said opening.

8. The device of claim 7 wherein said sleeve has an hourglass shape adapted to receive tubing of varying sizes.

9. The device of claim 3 wherein each sealing element includes a slit connected to said opening and said opening is sized to allow said sealing elements to be moveable on the tubing.

10. The device of claim 3 further including a slit in each of said sealing elements, said slit connected to said opening and forms first and second sections in said sealing elements.

11. The device of claim 10 further including a strap connected to said first section, said strap adapted to releasably connect to said second section and to compress said sections together.

12. The device of claim 10 further including a linear segment connected to said sealing elements, said linear segment adapted to urge said sealing elements toward each other.

13. The device of claim 12 wherein said sealing elements include sidewalls and said linear segment is adapted to urge said sidewalls toward each other.

14. The device of claim 10 further including a bridge formed by two opposing arms connected to said sealing elements to form a triangular opening adapted to receive a nose of a patient.

15. The device of claim 3 further including a bridge formed by two opposing arms connected to said sealing elements.

16. A method of creating a seal between a nasal cannula, a positive pressure ventilation mask and patient comprising the steps of: a providing a plurality of sealing elements, said sealing elements including an opening;
inserting a nasal cannula in said opening;
positioning said sealing elements on the tubing in a location to engage the edges of a mask; and
placing a mask on said patient where edges of said mask engage and compresses said sealing elements to form a seal between said sealing elements and the patient and between the mask and the sealing elements while the tubing is located in the sealing elements.

17. The method of claim 16 further including a linear segment connected to said sealing elements, said linear segment adapted to urge said sealing elements toward each other.

18. The method of claim 16 further including a bridge formed by two opposing arms connected to said sealing elements to form a triangular opening, adapted to receive a nose of a patient.

19. The device of claim 1 wherein said sealing elements are ovaloid in shape.

20. The device of claim 1 wherein said sealing elements are complimentary in shape to the edges of the mask.
